(19) United States
(12) Patent Application Publication
(10) Pub. No.: US 2025/0121162 A1
(43) Pub. Date: Apr. 17, 2025

(54) EXPANDABLE SHEATH WITH TRAPPING DILATOR

(71) Applicant: ABIOMED, Inc., Danvers, MA (US)

(72) Inventor: Robert Swierczek, Danvers, MA (US)

(73) Assignee: ABIOMED, Inc., Danvers, MA (US)

(21) Appl. No.: 18/912,774

(22) Filed: Oct. 11, 2024

Related U.S. Application Data
(60) Provisional application No. 63/590,036, filed on Oct. 13, 2023.

(51) Int. Cl. A61M 25/01; A61M 29/00

(52) U.S. Cl. CPC A61M 25/0113; A61M 29/00

(57) ABSTRACT

An expandable introducer sheath with a dilator configured to trap the distal tip of the introducer sheath. In some examples, the dilator may have a tip configured to slide relative to the body of the dilator, such that moving the dilator body in the proximal direction relative to the dilator tip reveals an area in which the outer diameter of the dilator transitions to an area of reduced diameter, and moving the dilator body in the distal direction relative to the dilator tip may enable the distal tip of the introducer sheath to become trapped. Likewise, in some examples, the dilator may be equipped with a collet configured to open and close as the dilator tip slides relative to the body of the dilator.

### CROSS-REFERENCE TO RELATED APPLICATIONS

This application claims priority to and the benefit of U.S. Provisional Application No. 63/590,036 which was filed on Oct. 13, 2023 and is incorporated by reference herein in its entirety.

### BACKGROUND

Intracardiac heart pump assemblies can be introduced into the heart either surgically or percutaneously and used to deliver blood from one location in the heart or circulatory system to another location in the heart or circulatory system. For example, when deployed in the heart, an intracardiac pump can pump blood from the left ventricle of the heart into the aorta, or pump blood from the inferior vena cava into the pulmonary artery. Intracardiac pumps can be powered by a motor located outside of the patient's body (and accompanying drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

In one approach, an intracardiac blood pump may be inserted by a catheterization procedure through the femoral artery using a sheath, such as a peel away introducer sheath. The sheath can alternatively be inserted in other locations suitable for delivery of a pump for supporting either the left or right side of the heart, such as in the femoral vein. The introducer sheath can be inserted into the femoral artery (or other vein or artery) through an arteriotomy, in order to create an insertion path for the pump assembly. A portion of the pump assembly may then be advanced through an inner lumen of the introducer and into the artery. Once the pump assembly has been inserted, the introducer sheath may be peeled away, so that a repositioning sheath can then be advanced over the pump assembly and into the arteriotomy. Replacing the introducer sheath with the repositioning sheath during insertion of a medical device may reduce limb ischemia and bleeding at the insertion site in the skin (and/or at the insertion site within the vessel) because of better fixation of the sheath to the patient when used with a hemostatic valve.

As tear-away introducer sheaths are generally not radially expandable, the inner diameter of the sheath must be large enough to accommodate the largest diameter portion of whatever device it is to be used with, such as the pump head of an intracardiac blood pump system. Thus, a peel-away introducer may create an opening that has an outer diameter wider than necessary to allow another narrower portion of the medical device to remain within the arteriotomy, such as the catheter portion of an intracardiac blood pump system. As a result, in some cases, the introducer sheath may be peeled or torn away after passage of the largest portion of the medical device, so that it can be replaced with a lower-profile repositioning sheath.

However, removing the introducer sheath by peeling it away may present various challenges. For example, if the introducer tears too easily and/or prematurely, it may lead to bleeding or vascular complications. Likewise, some peel-away introducer sheaths may require excessive force to tear away for removal. If a physician applies too much force, when the introducer finally tears, the physician may inadvertently shift the position of the pump within the heart. Further, peel-away configurations may complicate the design of the hemostatic valve located in the hub of the introducer, as these may also need to be configured to break away. In addition, even when a peel-away introducer sheath operates as intended, its use may nevertheless stretch the arteriotomy such that a larger vessel opening remains after the system is removed, which may in turn complicate vessel closure.

Some other medical introducers have expandable sheath bodies which may expand radially to allow passage of percutaneous devices into the patient's vasculature. These expandable introducers are generally for relatively short-term use and may be designed to prevent thrombosis between the sheath body and an indwelling catheter. These introducers are inserted having inner diameters smaller than the outer diameter of the device being introduced. The introducers expand to allow passage of the device through the sheath and into the vasculature and then may shrink again after the device has passed. Because these existing expandable introducers are intended for relatively short-term use, clot formation on the outside of the introducer sheath may be unlikely. However, if left in for longer periods of time (e.g., >1 hour, >2 hours, >6 hours, >1 day, >2 days, >1 week), clots may form on the outside surface of the expandable sheath, and risk being dislodged into the blood stream at a later time. Additionally, some commercially available expandable sheaths are completely flexible and therefore do not provide any rigidity within their structure thereby leading to kinking or buckling during insertion or withdrawal of a percutaneous medical device.

### BRIEF SUMMARY

The present technology relates to an expandable introducer sheath with a dilator configured to trap the distal tip of the introducer sheath. In some aspects of the technology, the distal end of the dilator may have a tip configured to slide relative to the body of the dilator, such that moving the dilator body in the proximal direction relative to the dilator tip reveals an area in which the outer diameter of the dilator transitions (e.g., through a step or taper) to an area of reduced diameter. The dilator may be further configured such that moving the dilator body in the distal direction relative to the dilator tip will enable the distal tip of the introducer sheath to become trapped. For example, the dilator may be configured to trap the distal tip of the introducer sheath between a proximal surface of the dilator tip and a tapered transition area proximal to the area of reduced diameter.

Likewise, in some aspects of the technology, the dilator may be further equipped with a collet configured to open and close as the dilator tip slides relative to the body of the dilator. For example, the dilator tip may be configured such that moving the dilator tip in the distal direction relative to the dilator body will both reveal an area in which the outer diameter of the dilator transitions (e.g., through a step or taper) to an area of reduced diameter and also allow the collet to expand radially. Conversely, the dilator may be configured such that moving the dilator body in the distal direction relative to the dilator tip will cause the dilator tip to slide over a portion of the area of reduced diameter and a portion of the collet, causing the collet to contract radially. This contraction of the collet may thus cause the collet to close over the distal tip of the introducer sheath, thus helping to trap the distal tip of the introducer sheath. For example, the collet may be configured to press the distal tip of the introducer sheath against a tapered transition area proximal to the area of reduced diameter.

Once the distal tip of the introducer sheath is trapped by the dilator as discussed above, tension may then be applied to the expandable sheath to further reduce its outer diameter and achieve a lower profile during insertion into a patient. In some aspects of the technology, the dimensions of the introducer sheath and dilator may be configured such that, when the distal tip of the introducer sheath is trapped and the sheath has been tensioned as discussed above, there will be a substantially smooth transition between the dilator tip and the body of the introducer sheath. Further, in some aspects, the distal tip of the introducer sheath may be tapered and/or may have an internal step configured to increase the likelihood that the distal tip will remain within the area of reduced diameter as the dilator tip is moved in the proximal direction and thus become trapped.

Notably, in some aspects of the technology, the trapping dilator assemblies of the present technology may enable a sheath tip to be loaded into the dilator tip and trapped without the use of specialized tools. This may make the assemblies of the present technology easier to use, and less prone to operator error. In addition, this may allow an operator to insert an expandable introducer sheath into a patient, release the sheath tip and remove the dilator assembly, and later reinsert the dilator into the introducer sheath and trap the sheath tip while the sheath remains in the patient (e.g., as may become necessary if the sheath tip needs to be repositioned).

Also described herein is a method for advancing a sheath using a dilator. According to the method, an expandable sheath having a proximal end, a distal end, and a lumen running from the proximal end to the distal end is joined to a dilator having a cylindrical body, a tapered tip, and a trapping mechanism between the body and the tapered tip. The trapping mechanism has a proximal section that includes a first cylindrical section with a first outer diameter; a second cylindrical section with a second outer diameter that is less than the first diameter; a first transitional section between the first cylindrical section and the second cylindrical section; and a collet having one or more shoulders connected to the second cylindrical section, and one or more arms extending proximally from the one or more shoulders. The trapping mechanism also has a distal section having a third cylindrical section with an inner diameter that is greater than the second outer diameter. According to the method the expandable sheath and the dilator are joined by inserting the dilator into the proximal end of the expandable sheath, sliding the proximal section of the trapping mechanism relative to the distal section of the trapping mechanism, compressing one or more arms of the collet by the third cylindrical section when the proximal section of the trapping mechanism is moved in a distal direction relative to the distal section of the trapping mechanism, and thereby trapping the expandable sheath between the one or more arms of the collet and a surface of the first transitional section when the proximal section of the trapping mechanism is moved in a distal direction relative to the distal section of the trapping mechanism.

In one aspect of the method, a sheath hub is coupled to the distal end of the expandable sheath and a dilator hub is coupled to a distal end of the body of the dilator. The dilator hub may have one or more latches, and the method further includes locking the dilator hub to the sheath hub.

### BRIEF DESCRIPTION OF DRAWINGS

FIG. 1 shows an exemplary sheath assembly, in accordance with aspects of the technology.

FIG. 2 shows an exemplary sheath assembly, in accordance with aspects of the technology.

FIG. 3 shows an exemplary dilator assembly, in accordance with aspects of the technology.

FIG. 4 shows a close-up view of the trapping mechanism of the exemplary dilator assembly of FIG. 3, in accordance with aspects of the technology.

FIGS. 5A and 5B show cross-sectional side views of the trapping mechanism of the exemplary dilator assembly of FIG. 3 in engagement with an exemplary sheath, in accordance with aspects of the technology.

### DETAILED DESCRIPTION

The present technology will now be described with respect to the following exemplary systems and methods. Reference numbers in common between the figures depicted and described below are meant to identify the same features.

Although certain features of the present technology may be described in connection with an intracardiac heart pump system, it will be understood that the components and features described herein may be combined with one another in any suitable manner and may be adapted for use with any suitable type of medical device. For example, the present technology may be used to introduce electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices.

The systems, methods, and devices described herein may provide an expandable sheath assembly for the insertion of a medical device (e.g., an intracardiac heart pump) into a blood vessel through a vessel aperture, and an associated dilator assembly configured to trap a distal end of the expandable sheath body. The expandable sheath and dilator assemblies of the present technology may be especially advantageous over existing expandable sheath and dilator assemblies for patients with coronary artery disease (CAD) and peripheral artery disease, presenting with calcification and tortuosity of arteries, making delivery of introducer sheaths and catheters difficult. The expandable sheath and dilator assemblies of the present technology may also be easier to insert than traditional assemblies because of their reduced insertion profile, increased flexibility, reduced friction, and reduced risk of kinking under loads. For example, the reduced insertion diameter and smooth profile may minimize insertion related complications, minimize stretching and load on the vessel opening, and minimize the risk of limb ischemia. The structure of the sheath body described herein may further provide sufficient axial stiffness for pushability and buckling resistance, while maintaining bending flexibility and kink resistance, and reducing frictional force to prevent “finger trapping.” Moreover, the structures of the sheath body described herein may provide an improvement over existing introducer sheath bodies by having a smooth inner surface with a thin coating thickness reducing the force required to expand the sheath (compared to the force required to expand a sheath having a coating without any bias), and/or by having a smooth outer surface reducing the risk of thrombus formation during use over longer durations while at the same time enabling the sheath to expand and contract as desired and reducing friction between the sheath body and devices being inserted through it. Furthermore, the structure of the sheath body described herein may be configured to interface with a trapping dilator assembly, such that the sheath body can be held in place, and optionally held in tension, during insertion into a patient's vasculature. By configuring the sheath for use with a trapping dilator assembly, it may be possible to make the sheath body thinner, more flexible, and/or less axially stiff than if it would otherwise need to be for insertion over a non-trapping dilator.

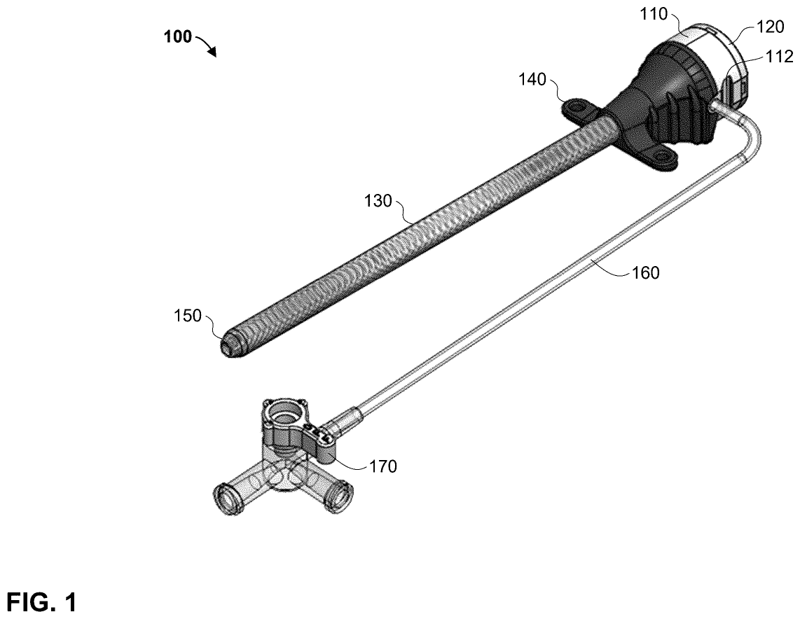

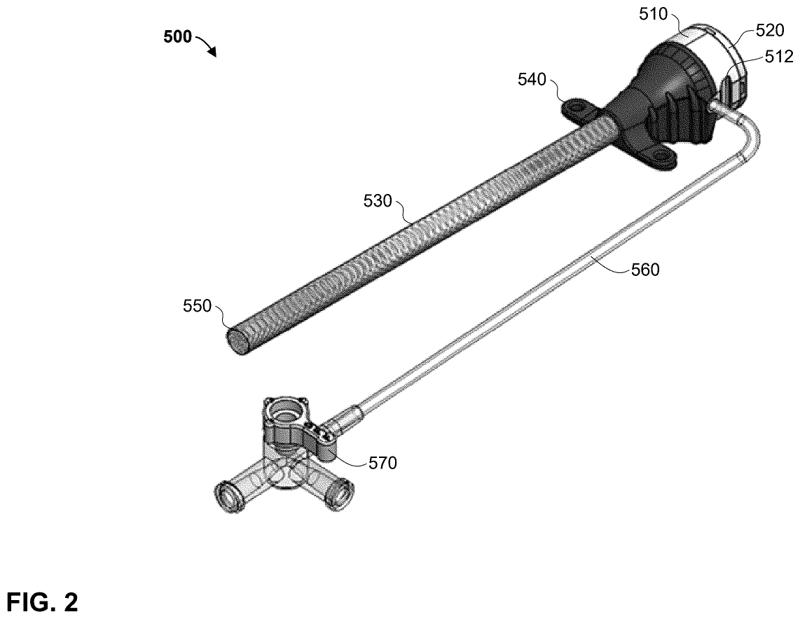

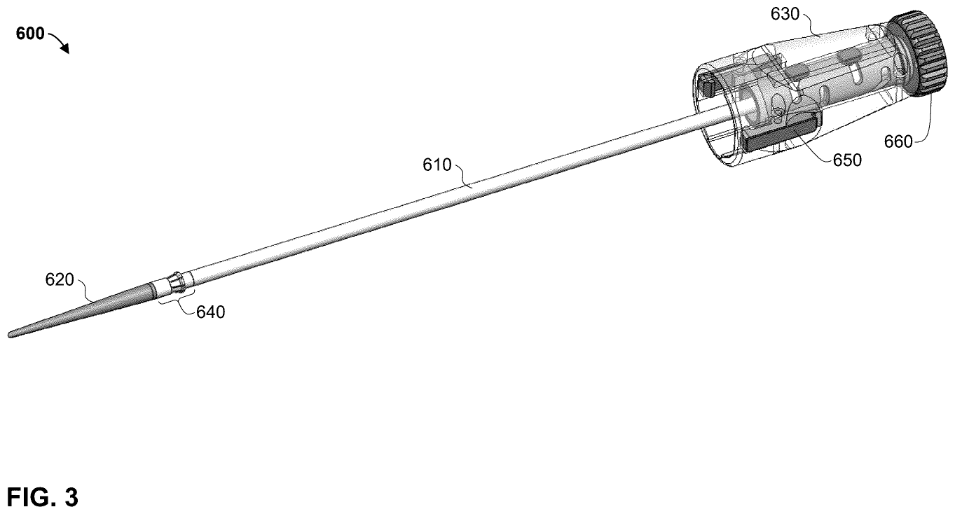

FIG. 1 shows an exemplary sheath assembly 100, in accordance with aspects of the technology. The sheath assembly has a hub 110 that may lock the sheath in position once inserted. The hub 110 may work in concert with the cap 120 to secure the sheath body 130 in position. In the example of FIG. 1, the hub 110 also has detents 112 (only one of which is visible) which may aid in attaching hub 110 to the hub of a dilator (e.g., dilator hub 630 of FIG. 3) as described further below. Further, a butterfly/suture pad 140 may be included to aid in attaching the sheath assembly 100 to the patient (e.g., by suturing the assembly to the patient). As can be seen, in the example of FIG. 1, the distal end of the sheath body 130 is shown having a tapered sheath tip 150. However, in some aspects of the technology, the sheath tip 150 may not be tapered, and may thus have an outer diameter equal to that of the sheath body 130 (e.g., as shown in FIG. 2). In cases where the sheath tip 150 is tapered, it may have a straight linear taper, convex taper, concave taper, or a taper composed of one or more straight, convex, and/or concave sections. The sheath tip 150 may also be of any suitable length. In some aspects of the technology, sheath tip 150 may be between 0.1 mm and 5 mm in length. In the present description, “proximal” is used to indicate a direction pointing towards an operator of the device and away from the patient, or an end of the device that is closer to the operator than to the patient; “distal” is used to indicate used to indicate a direction pointing away from an operator of the device and towards the patient, or an end of the device that is closer to the patient than to the operator. Accordingly, the proximal end of the sheath assembly 100 is at the end with the hub 110 and cap 120 and the distal end of the assembly is at the end with sheath tip 150.

In the example of FIG. 1, fluids may be introduced into the sheath assembly 100 via a sidearm channel 160, and fluid flow into the sidearm channel 160 may be controlled by a stopcock 170. A hemostatic valve (not shown) may also be included within hub 110, and may be configured to prevent blood from leaking outside of the patient during insertion and/or removal of an intracardiac blood pump or other components. Although any suitable hemostatic valve may be employed, examples are described and illustrated in U.S. Published Application No. 2021/0146111 A1, which is hereby incorporated by reference. In addition, in some aspects of the technology, the hub 110 may further include a foam insert (not shown) placed proximal to the hemostatic valve, and the foam insert may be soaked with a lubricant such as silicone so that components will be lubricated as they are inserted through the foam and into the sheath body 130.

The expandable sheath body 130 may comprise at least a frame and a coating. A coating may be applied to the outer surface of the sheath body 130 to facilitate passage inside the patient, known as an outer-diameter biased approach. This coating may be formed from any suitable material or combination of materials. For example, in some aspects of the technology, the coating may include a thermoplastic polymer (“TPU”). This outer-diameter biased coating may advantageously provide a smooth outer surface which reduces the risk of clot formation and minimizes friction when inserting a device through the expandable sheath. For example, the use of a smooth outer surface may advantageously minimize the risk of clots forming on the surface of the expandable sheath body 130, and a corrugated inner surface may minimize the surface area of the expandable sheath in contact with a device being pushed through, thereby minimizing associated friction forces. In some aspects of the technology, a corrugated inner surface may be provided through the use of a braided material as the frame of the expandable sheath body 130.

The braided material may also be formed from any suitable material. For example, a braided material may include or be composed of one or more strands of a flexible metal such as Nitinol.

In some aspects of the technology, an additional lubricious coating may be applied to the inner and/or outer surfaces of sheath body 130, i.e., covering the exposed portions of the frame and the coating. An outer-diameter biased coating may further advantageously provide for a thin coating thickness, such that a relatively smaller force may be required to expand the sheath body 130 compared to a force required to expand a sheath having a coating without any bias. And outer-diameter biased coating may also advantageously allow the sheath frame to expand and contract as desired, i.e., the outer-diameter biased coating may not immobilize the frame at a fixed diameter if the coating thickness is sufficiently thin to avoid encapsulating the portions of the frame where frame elements intersect. For example, for a braided frame having braided elements in an over-under braid pattern and an outer-diameter biased coating, an outer diameter biased coating may be made thin enough that it does not encapsulate an overlap of braided elements, and thus does not extend to the braided elements located under other braided elements in the over-under braided pattern.

The expandable sheath body 130 and sheath tip 150 may be formed in a variety of ways, including using the configurations and methods of manufacture described in U.S. Patent Publication No. 2019/0247627A1, U.S. Patent Publication No. 2020/0054861A1, and/or U.S. Patent Publication No. 2018/0256859A1, which are incorporated by reference herein. For example, the expandable sheath body 130 (and sheath tip 150) may be manufactured using thermal bonding or an outer-diameter biased dipping, which may provide the sheath body 130 with a smooth outer surface while retaining a desired spring-like expandable nature. Specific details of the possible configurations for sheath body 130 and methods of manufacturing them are included in the referenced published applications, and are thus not repeated in full herein.

By employing a frame and coating assembly as described above and in the referenced applications, the expandable sheath body 130 may be configured to expand and collapse while also remaining resistant to kinking. This may enable the sheath body 130 to expand to permit insertion or recovery of the medical device, and then return to its original shape after deformation. In addition, configuring the expandable sheath for compatibility with a trapping dilator assembly (e.g., dilator assembly 600 of FIG. 3) may aid in inserting the sheath, removing the dilator, and/or reinserting the dilator and recapturing the sheath tip after the sheath is within the patient (e.g., as may be necessary to reposition the sheath within the patient).

Moreover, the expandable nature of sheath body 130 may eliminate the need to use multiple sheaths, such as a peel-away introducer sheath and a repositioning sheath, for the introduction of a medical device (e.g., an intracardiac heart pump) into the vessel opening (e.g., arteriotomy). In that regard, once the expandable sheath body 130 is positioned within the patient's vasculature, it may be left in place even after the medical device is removed in order to maintain access to a vessel should it be needed again. This may improve the efficiency of any medical procedure, and simplify the process of inserting medical devices into the patient, as it removes the need to peel away an introducer sheath and replace it with a repositioning sheath every time access to the vessel opening is required. This may also lower the risk of the medical procedure by removing the risk that the peel-away introducer sheath may tear prematurely and/or that the introduced medical device may be inadvertently shifted while the introducer sheath is peeled away and replaced with a repositioning sheath. Notwithstanding the foregoing, the expandable sheaths described herein may still be used in conjunction with a repositioning sheath.

Although the sheath tip 150 may have an internal step feature, in some aspects of the technology, the internal surface of the sheath tip 150 may be tapered at the same angle as the external surface such that a constant wall thickness is obtained. In addition, in some aspects of the technology, sheath tip 150 may not be tapered at all (e.g., as shown in FIG. 2). For example, sheath tip 150 may have the same outer diameter as at least a portion of the sheath body 130 when the sheath is in a relaxed state. In such cases, the outer diameter of the sheath tip 150 in its relaxed state may be configured to be smaller than the outer diameter of the first cylindrical section 641 or even the second cylindrical section 643 of the trapping mechanism 640, such that the sheath tip 150 will still tend to hug the second cylindrical section 643 and/or the first transitional section 642 prior to being trapped by the closing of the trapping mechanism 640. Further, in some aspects of the technology, although the sheath tip 150 may be configured to have the same outer diameter as at least a portion of the sheath body 130 in its relaxed state, the sheath tip 150 may be configured to be less elastic than the sheath body 130 in order to increase the likelihood that the sheath tip 150 will tend to hug the second cylindrical section 643 and/or the first transitional section 642 prior to being trapped by the closing of the trapping mechanism 640.

In some aspects of the technology, some or all of the internal surfaces of sheath tip 150 may be textured or otherwise configured to reduce friction and stiction between those surfaces and other devices that pass through it, e.g., the dilator tip 620, interventional devices introduced through sheath assembly 100 such as intracardiac heart pumps, etc. Texturing may be applied to surfaces of sheath tip 150 by forming it using a mandrel which itself has been textured through machining, sand-blasting, shot peening, chemical etching, laser surface texturing, etc. In that regard, in some aspects of the technology, the internal surfaces of sheath tip 150 may be cross-hatched, knurled, or dimpled. In some aspects of the technology, the internal surfaces of sheath tip 150 may have a pattern composed of dashed or continuous lines, which may extend in any direction, e.g., longitudinally, circumferentially, or any angle therebetween. In some aspects of the technology, the internal surfaces of sheath tip 150 may have a pattern of lines that are curvilinear, sinusoidal, saw-toothed, or any combination thereof, and which may extend in any direction, e.g., longitudinally, circumferentially, or any angle therebetween. In some aspects of the technology, the internal surfaces of sheath tip 150 may have one or more raised or recessed grooves, which may extend in any direction, e.g., longitudinally, circumferentially, or any angle therebetween. Likewise, in some aspects of the technology, the internal surfaces of sheath tip 150 may be coated or comprised of materials that reduce friction or stiction. For example, the internal surfaces of sheath tip 150 may have a lubricious coating, or may be formed from a material with a suitably low coefficient of friction, e.g., PTFE. The internal surfaces of sheath tip 150 may incorporate any combination of the different options described above, including a combination of textured features as well as lubricious coatings and/or low-friction materials.

FIG. 2 shows an exemplary sheath assembly 500, in accordance with aspects of the technology. The sheath assembly has a hub 510 that may lock the sheath in position once inserted. The hub 510 may work in concert with the cap 520 to secure the sheath body 530 in position. In the example of FIG. 2, the hub 510 also has detents 512 (only one of which is visible) which may aid in attaching hub 510 to the hub of a dilator (e.g., dilator hub 630 of FIG. 3) as described further above and below. Further, a butterfly/suture pad 540 may be included to aid in attaching the sheath assembly 500 to the patient (e.g., by suturing the assembly to the patient). As can be seen, in the example of FIG. 2, the distal end of the sheath body 530 is shown having a straight sheath tip 550 with an outer diameter equal to that of the sheath body 530. However, in some aspects of the technology, the sheath tip 550 may be tapered (e.g., as shown in FIG. 1). In cases where the sheath tip 550 is tapered, it may have a straight linear taper, convex taper, concave taper, or a taper composed of one or more straight, convex, and/or concave sections. The sheath tip 550 may also be of any suitable length. In some aspects of the technology, sheath tip 150 may be between 0.1 mm and 5 mm in length. Likewise, in some aspects of the technology, the sheath tip 550 may have no length, such that the sheath tip 550 simply indicates the distal end of the sheath body 530. Here as well, the proximal end of the sheath assembly 500 is at the end with the hub 510 and cap 520 and the distal end of the assembly is at the end with sheath tip 550.

In the example of FIG. 2, fluids may be introduced into the sheath assembly 500 via a sidearm channel 560, and fluid flow into the sidearm channel 560 may be controlled by a stopcock 570. A hemostatic valve (not shown) may also be included within hub 510, and may be configured to prevent blood from leaking outside of the patient during insertion and/or removal of an intracardiac blood pump or other components. Although any suitable hemostatic valve may be employed, examples are described and illustrated in U.S. Published Application No. 2021/0146111 A1, which is hereby incorporated by reference. In addition, in some aspects of the technology, the hub 510 may further include a foam insert (not shown) placed proximal to the hemostatic valve, and the foam insert may be soaked with a lubricant such as silicone so that components will be lubricated as they are inserted through the foam and into the sheath body 530.

Here as well, the expandable sheath body 530 may comprise at least a frame and a coating, and may be configured in any of the ways described above with respect to sheath body 110 of FIG. 1. Likewise, the expandable sheath body 530 and sheath tip 550 may be formed in any of the ways described above with respect to sheath body 130 and sheath tip 150 of FIG. 1. Again, by employing a frame and coating assembly as described above and in the referenced applications, the expandable sheath body 530 may be configured to expand and collapse while also remaining resistant to kinking. This may enable the sheath body 530 to expand to permit insertion or recovery of the medical device, and then return to its original shape after deformation. In addition, configuring the expandable sheath for compatibility with a trapping dilator assembly (e.g., dilator assembly 600 of FIG. 3) may aid in inserting the sheath, removing the dilator, and/or reinserting the dilator and recapturing the sheath tip after the sheath is within the patient (e.g., as may be necessary to reposition the sheath within the patient).

Here as well, the expandable nature of sheath body 530 may eliminate the need to use multiple sheaths, such as a peel-away introducer sheath and a repositioning sheath, for the introduction of a medical device (e.g., an intracardiac heart pump) into the vessel opening (e.g., arteriotomy). In that regard, once the expandable sheath body 530 is positioned within the patient's vasculature, it may be left in place even after the medical device is removed in order to maintain access to a vessel should it be needed again. This may improve the efficiency of any medical procedure, and simplify the process of inserting medical devices into the patient, as it removes the need to peel away an introducer sheath and replace it with a repositioning sheath every time access to the vessel opening is required. This may also lower the risk of the medical procedure by removing the risk that the peel-away introducer sheath may tear prematurely and/or that the introduced medical device may be inadvertently shifted while the introducer sheath is peeled away and replaced with a repositioning sheath. Notwithstanding the foregoing, the expandable sheaths described herein may still be used in conjunction with a repositioning sheath.

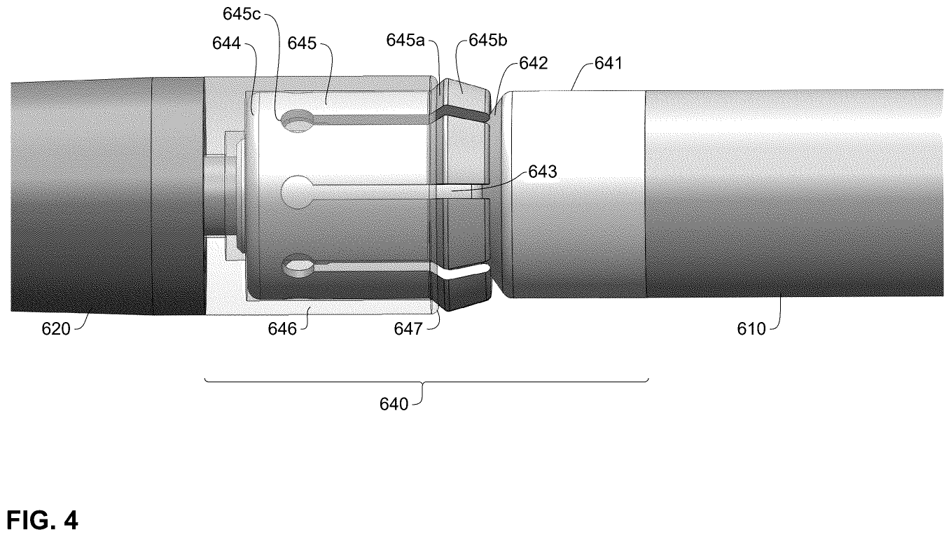

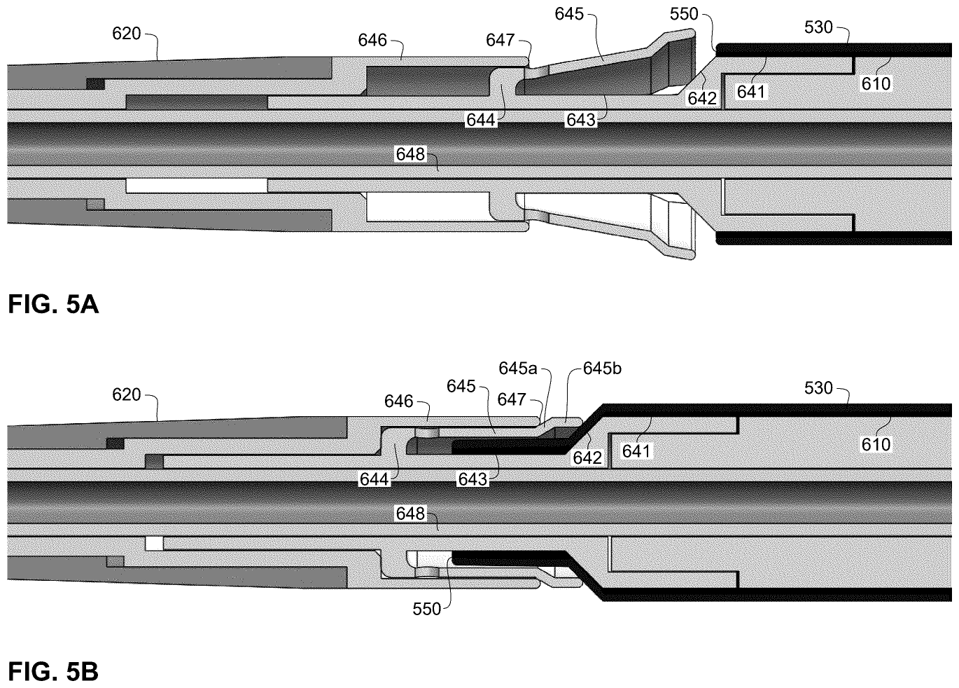

FIG. 3 shows an exemplary dilator assembly 600, in accordance with aspects of the technology. In addition, FIG. 4 shows a close-up view of the trapping mechanism 640 of the exemplary dilator assembly of FIG. 3, and FIGS. 5A and 5B show cross-sectional side views of the trapping mechanism 640 of the exemplary dilator assembly of FIG. 3 in engagement with an exemplary sheath.

As shown in FIGS. 3-5B, the dilator assembly 600 may include a dilator having a dilator body 610, a dilator tip 620 at its distal end, and a trapping mechanism 640 between the dilator body 610 and the dilator tip 620. The dilator tip 620 may be tapered as it approaches its distal end, to facilitate insertion into the patient's vasculature. The dilator assembly 600 may further include a dilator hub 630 at its proximal end, having toothed latches 650 configured to secure it to the hub of an introducer sheath (e.g., hub 110 of the introducer sheath assembly 100, hub 510 of the introducer sheath assembly 500), and knob 660 configured to cause the trapping mechanism 640 to open and close when rotated.

Dilator tip 620, trapping mechanism 640, and dilator body 610 may be made of any suitable material. In some aspects of the technology, dilator tip 620 may be formed of a flexible material such as polyether block amide (“PEBA”) with a durometer hardness of 40D. In some aspects of the technology, dilator tip 620 may be formed of other flexible materials such as PEBA with other hardness ratings, silicone, thermoplastic polyurethane (“TPU”), or thermoplastic elastomer (“TPE”). In some aspects of the technology, dilator tip 620 may further include hydrophilic lubricious coating such as polyvinylpyrrolidone (“PVP”) or hyaluronic acid (“HA”), or a hydrophobic coating such as silicone or polytetrafluoroethylene (“PTFE”). In some aspects of the technology, dilator tip 620 may have no coating.

In some aspects of the technology, dilator body 610 may be formed of a semi-rigid material such as PEBA with a durometer hardness of 70D. In some aspects of the technology, dilator body 610 may be other semi-rigid materials such as PEBA with other hardness ratings, polyethylene, polypropylene, or polyurethane.

In some aspects of the technology, the trapping mechanism 640 may be formed in whole or in part of metals such as 304 stainless steel, 316 stainless steel, and/or Nitinol. Likewise, in some aspects of the technology, the trapping mechanism 640 may be formed in whole or in part of a suitable polymer material, such as polyether ether ketone (“PEEK”), acrylonitrile butadiene styrene (“ABS”), and/or polycarbonate. In some aspects of the technology, trapping mechanism 640 may be fully or partially coated, such as with a polymer. In some aspects of the technology, trapping mechanism 640 may have a coating that is between 0.025 and 0.2 mm. In some aspects of the technology, trapping mechanism 640 may have a coating with a durometer hardness of between 40 A and 70 D. In some aspects of the technology, trapping mechanism 640 may have a coating with a coefficient of friction that is greater than that of stainless steel and/or the material chosen for the dilator tip 620 or dilator body 610. In some aspects of the technology, trapping mechanism 640 may have no coating.

In the example of FIGS. 3-5B, the trapping mechanism 640 is shown having a proximal section coupled to the dilator body 610, and a distal section coupled to the dilator tip 620, with the proximal section being configured to slide at least partially inside the distal section. The proximal section and distal section may additionally be supported from within by a central rod or tube 648 (not visible in FIG. 2 or 3, but shown in FIGS. 5A and 5B). For example, in some aspects of the technology, the distal section of the trapping mechanism 640 may be fixedly coupled to the central rod or tube 648 (e.g., using an interference fit, adhesive, welding, or another fixation method) and the proximal section of the trapping mechanism 640 and the dilator body 610 may be configured to have enough clearance such that they will slide over the central rode or tube 648 when the knob 660 is rotated. In such cases, the central rod or tube 648 may be coupled to a portion of the dilator hub 630 that moves proximally and distally when the knob 660 is rotated, while the dilator body 610 is coupled to portion of the dilator hub 630 that remains stationary when the knob 660 is rotated.

In some aspects of the technology, all or a portion of the central rod or tube 648 may be solid. Likewise, in some aspects of the technology, all or a portion of the central rode or tube 648 may be hollow. For example, where the entirety of the central rod or tube 648 is hollow, it may form all or a portion of a continuous lumen running through the entire dilator assembly 600, such that the dilator may be introduced into a patient over a guide wire that has already been introduced into the patient's vasculature.

In the example of FIGS. 3-5B, the proximal section of the trapping mechanism 640 includes a first cylindrical section 641 having a first outer diameter, a first transitional section 642, a second cylindrical section 643 having a second outer diameter smaller than the first outer diameter, and a collet composed of one or more shoulders 644 and one or more arms 645. The combination of these sections forms an area of reduced diameter into which the dilator tip 550 may be trapped, as shown in FIG. 5B.

Any suitable profiles, shapes, and absolute or relative dimensions may be used for each of sections 641-645. Thus, in some aspects of the technology, the first outer diameter may be equal or substantially equal to the outer diameter of the portion of the dilator body 610 to which it abuts. Likewise, in some aspects, the first outer diameter may be equal or substantially equal to the outer diameter of a portion of shoulder 644. Further, although the first transitional section 642 is shown being chamfered, any suitable type of transition may be used. Thus, the profile of the first transitional section 642 may include a 90-degree step, a straight linear taper of any angle, a convex taper of any contour, a concave taper of any contour, or any suitable combination thereof. Likewise, the first transitional section 642 may include multiple steps, multiple straight linear tapers of different angles, multiple convex tapers of different contours, multiple concave tapers of different contours, or any suitable combination thereof.

In the example of FIGS. 3-5B, the distal section of the trapping mechanism 640 includes a third cylindrical section 646 having a third outer diameter, and an inner diameter that is greater than the second outer diameter. In addition, in this example, the proximal edge 647 of the third cylindrical section 646 is rounded. However, in some aspects of the technology, the proximal edge 647 of the third cylindrical section 646 may have another profile, such as a straight edge, a chamfer, etc. Here as well, any suitable profiles and absolute or relative dimensions may be used for sections 646 and 647. Thus, as shown in FIGS. 5A and 5B, the third outer diameter of the third cylindrical section 646 may be slightly larger than the first outer diameter of the first cylindrical section 641, such that when an introducer sheath is trapped in the trapping mechanism 640 and the introducer sheath is tensioned so that it is drawn down against the first cylindrical section 641 (e.g., as shown in FIG. 5B), the outer diameter of the introducer sheath will be substantially the same as the third outer diameter of the third cylindrical section 646, thus providing a generally smooth profile to the combined sheath/dilator assembly. However, in some aspects of the technology, the third outer diameter of the third cylindrical section 646 may be the same as, or smaller than, the first outer diameter of the first cylindrical section 641.

In the example of FIGS. 5A and 5B, the sheath tip 550 is not tapered, and thus has an outer diameter equal to that of the sheath body 530 when the sheath is in a relaxed state. Although FIGS. 5A and 5B do not show any distinction between the sheath tip 550 and the sheath body 530, the sheath tip 550 may have a different configuration than the sheath body 530. For example, in some aspects of the technology, the sheath body 550 may comprise a frame and a coating of one or more layers of polymer (e.g., as discussed above with respect to FIGS. 1 and 2), whereas the frame may not extend into the sheath tip 530 or may extend only partially into the sheath tip 530, such that a portion of the sheath tip 530 is only composed of one or more layers of polymer. Likewise, in some aspects, the sheath body 550 may comprise a frame and a coating of one or more layers of polymer, and the sheath tip 530 may include a coating of different or additional polymer layers. Further, in some aspects, the sheath tip 550 may have an internal lumen equal to that of the sheath body 530 when the sheath is relaxed, but may be thicker than the sheath body 530 such that the outer diameter of the sheath tip 550 is larger than that of the sheath body 530 when the sheath is in a relaxed state. In all cases, any suitable thicknesses may be employed for the sheath body 530 and the sheath tip 550.

In addition, the sheath body 530 and sheath tip 550 may be configured with internal diameters that are equal to or smaller than the outer diameter of the dilator body 610, such that they will tend to conform to the dilator body 610 as shown in the example of FIG. 5A. This may thus aid the operator in properly positioning the sheath tip 550 prior to closing the trapping mechanism 640. In that regard, with the sheath and trapping mechanism arranged as shown in FIG. 5A, the operator may position the sheath for trapping by withdrawing the dilator proximally until the sheath tip 550 slides under the arms 645 of the collet. The operator may then close the trapping mechanism 640 (e.g., by turning knob 660), which will cause the distal section and proximal section of the trapping mechanism 640 to slide toward one another. As the distal section of the trapping mechanism 640 slides over the shoulder 644 and arms 645 of the collet, and third cylindrical section 646 will compress the arms 645 and cause them to pin a section of the sheath tip 550 (or the sheath body 530 depending on the level of insertion) against the first transitional section 642, thus trapping the sheath tip 550 (or the sheath body 530) as shown in FIG. 5B. As will be appreciated, the shoulder 644 and arms 645 of the collet may be configured to allow the collet to be opened and closed multiple times without experiencing fatigue or breakage. For example, the collet may be made of a superelastic material such as Nitinol and may be configured with rounded cutouts 645c between each arm 645 in order to allow the arms 645 to flex without creating cracks.

In some aspects of the technology, the internal surface of sheath tip 550 may be textured or otherwise configured to reduce friction and stiction between those surfaces and other devices that pass through it, e.g., the dilator tip 620, the third cylindrical section 646, the outer surface of arms 645, interventional devices introduced through sheath assembly 100 such as intracardiac heart pumps, etc. Texturing may be applied using any suitable method. For, example, texturing may be applied to sheath tip 550 by forming it using a mandrel which itself has been textured through machining, sand-blasting, shot peening, chemical etching, laser surface texturing, etc. In that regard, in some aspects of the technology, the internal surfaces of sheath tip 550 may be cross-hatched, knurled, or dimpled. In some aspects of the technology, the internal surfaces of sheath tip 550 may have a pattern composed of dashed or continuous lines, which may extend in any direction, e.g., longitudinally, circumferentially, or any angle therebetween. In some aspects of the technology, the internal surfaces of sheath tip 550 may have a pattern of lines that are curvilinear, sinusoidal, saw-toothed, or any combination thereof, and which may extend in any direction, e.g., longitudinally, circumferentially, or any angle therebetween. In some aspects of the technology, the internal surfaces of sheath tip 550 may have one or more raised or recessed grooves, which may extend in any direction, e.g., longitudinally, circumferentially, or any angle therebetween. Likewise, in some aspects of the technology, the internal surfaces of sheath tip 550 may be coated or comprised of materials that reduce friction or stiction. For example, the internal surfaces of sheath tip 550 may have a lubricious coating, or may be formed from a material with a suitably low coefficient of friction, e.g., PTFE. The internal surfaces of sheath tip 550 may incorporate any combination of the different options described above, including a combination of textured features as well as lubricious coatings and/or low-friction materials.

Likewise, in some aspects of the technology, one or both of the transitional section 642 and second cylindrical section 643 of the trapping mechanism may be textured or otherwise configured to increase friction and stiction between those surfaces and the sheath tip 550. Here as well, texturing may be applied to the surfaces of the first transitional section 642 and/or the second cylindrical section 643 in any suitable method. For, example, texturing may be applied to the surfaces of the first transitional section 642 and/or the second cylindrical section 643 by forming them using a mold which itself has been textured through machining, sand-blasting, shot peening, chemical etching, laser surface texturing, etc. In that regard, in some aspects of the technology, the surfaces of the first transitional section 642 and/or the second cylindrical section 643 may be cross-hatched, knurled, or dimpled. In some aspects of the technology, the surfaces of the first transitional section 642 and/or the second cylindrical section 643 may have a pattern composed of dashed or continuous lines, which may extend in any direction, e.g., longitudinally, circumferentially, or any angle therebetween. In some aspects of the technology, the surfaces of the first transitional section 642 and/or the second cylindrical section 643 may have a pattern of lines that are curvilinear, sinusoidal, saw-toothed, or any combination thereof, and which may extend in any direction, e.g., longitudinally, circumferentially, or any angle therebetween. In some aspects of the technology, the surfaces of the first transitional section 642 and/or the second cylindrical section 643 may have one or more raised or recessed grooves, which may extend in any direction, e.g., longitudinally, circumferentially, or any angle therebetween. Likewise, in some aspects of the technology, the surfaces of the first transitional section 642 and/or the second cylindrical section 643 may be coated or comprised of materials that increase friction or stiction. For example, the surfaces of the first transitional section 642 and/or the second cylindrical section 643 may have a gritty coating, or may be formed from a material with a suitably high coefficient of friction, e.g., unpolished stainless steel. Further, in some aspects of the technology, the first transitional section 642 and/or the second cylindrical section 643 may include a magnetic material (e.g., rare earth magnets such as neodymium or samarium-cobalt), and the sheath tip 550 may include a metallic ring (e.g., one or more threads of a magnetic stainless steel), so as to create an attraction between the sheath tip 550 and the first transitional section 642 and/or the second cylindrical section 643. The surfaces of the first transitional section 642 and/or the second cylindrical section 643 may incorporate any combination of the different options described above, including a combination of textured features as well as coatings, higher-friction materials, and/or magnetic materials.

Here as well, once the sheath tip 550 is trapped by the trapping mechanism 640 as shown in FIG. 5B, the sheath may then be tensioned by pulling the sheath hub 510 or the proximal end of the sheath body 530 away from the dilator tip 620, or by pushing the dilator tip 620 away from the sheath hub 510 or the proximal end of the sheath body 530. In some aspects of the technology, the sheath assembly 500 and the dilator assembly 600 may be dimensioned such that once the sheath tip 550 is trapped by the trapping mechanism 640, the sheath body 530 may be appropriately tensioned by simply locking the sheath hub 510 into the dilator hub 630. However, it may be achieved, tensioning the sheath body 530 will cause the sheath body 530 to elongate and narrow, which may advantageously reduce its insertion profile, and thus help to minimize patient complications (e.g., bleeding, vascular injury, high insertion forces). At this point, the dilator assembly 600 may be used to insert the sheath tip 550 and sheath body 530 into the patient's vasculature.

Once the sheath body 530 has been positioned as desired within the patient's vasculature, the trapping mechanism 640 may be opened (e.g., by turning knob 660 in the opposite direction used to close it), and the sheath body 530 may be pulled proximally so that sheath tip 550 slides back into the position shown in FIG. 5A. In addition, in some aspects of the technology, the sheath assembly 500 and dilator assembly 600 may be configured such that there will be enough tension on the sheath body 530 when the sheath hub 510 and dilator hub 630 are engaged and the sheath tip 550 is trapped, that the sheath tip 550 will withdraw back to the position shown in FIG. 5A if the trapping mechanism 640 is opened while the sheath hub 510 and dilator hub 630 remain locked together. In either case, once the sheath has returned to the position shown in FIG. 5A, the trapping mechanism 640 may be closed again without the sheath tip 550 becoming trapped. Then, the dilator hub 630 may be unlocked from the hub 510 of sheath assembly 500 by pressing the toothed latches 650, and the dilator assembly 600 may be retracted by pulling the dilator hub 630 in the proximal direction relative to the sheath hub 510. This retraction will cause the sheath tip 650 to slide over the closed trapping mechanism 640 and the dilator tip 620, until the dilator assembly 600 is fully removed from the patient. The sheath assembly 500 may then be used to introduce an intracardiac blood pump and/or other components into the patient's vasculature as discussed above. Notably, as sheath body 530 will no longer be in tension after the locking mechanism 640 is opened, sheath body 530 will be free to relax into a shorter and wider configuration that aids in removal of the dilator assembly 600. Likewise, as sheath body 530 will no longer be in tension after the dilator assembly 600 has been withdrawn, sheath body 530 will also be free to relax into a shorter and wider configuration that aids in insertion of an intracardiac blood pump and/or other components into the patient's vasculature.

Described herein is an apparatus, that may have an expandable sheath with a proximal end, a distal end, and a lumen running from the proximal end to the distal end; a dilator that may have a cylindrical body, a tapered tip, and a trapping mechanism between the body and the tapered tip, wherein the trapping mechanism may have a proximal section with a first cylindrical section with a first outer diameter; a second cylindrical section with a second outer diameter that is less than the first diameter; a first transitional section between the first cylindrical section and the second cylindrical section; and a collet having one or more shoulders connected to the second cylindrical section, and one or more arms extending proximally from the one or more shoulders. In a further aspect the trapping mechanism may have a distal section having a third cylindrical section with an inner diameter that is greater than the second outer diameter, wherein the dilator is configured to be inserted into the proximal end of the expandable sheath, the proximal section of the trapping mechanism is configured to slide relative to the distal section of the trapping mechanism, and the one or more arms of the collet are configured to be compressed by the third cylindrical section when the proximal section of the trapping mechanism is moved in a distal direction relative to the distal section of the trapping mechanism, and further wherein the trapping mechanism is configured to trap the expandable sheath between the one or more arms of the collet and a surface of the first transitional section when the proximal section of the trapping mechanism is moved in a distal direction relative to the distal section of the trapping mechanism.

In a further aspect, the distal end of the expandable sheath is tapered. In any of the above aspects, an internal surface of the distal end of the expandable sheath may include a step feature. The above aspects may further include a sheath hub coupled to the distal end of the expandable sheath; and a dilator hub coupled to a distal end of the body of the dilator having one or more latches configured to lock the dilator hub to the sheath hub.

In any of the above aspects, the expandable sheath may have an expandable frame and a material applied to the frame, wherein the material may optionally be composed of a polymer that may be a thermoplastic polyurethane. In a further aspect the frame may be formed from a braided material, wherein the braided material is optionally composed from strands of Nitinol. In a further aspect the expandable sheath may have a coating applied to the expandable frame and the material. In a further aspect, the coating may be a lubricious coating.

In any of the above aspects, the trapping mechanism may be formed from Nitinol and/or the collet may be formed from Nitinol. In an alternative aspect, the trapping mechanism may be formed from stainless steel. In another alternative aspect, the trapping mechanism may be formed from a polymer.

In any of the above aspects, the tapered tip of the dilator may be formed from a polymer that is optionally a polyether block amide.

Also described is a method for advancing a sheath using a dilator, including the following steps:
joining an expandable sheath having a proximal end, a distal end, and a lumen running from the proximal end to the distal end to a dilator comprising a cylindrical body, a tapered tip, and a trapping mechanism between the body and the tapered tip, wherein the trapping mechanism may have:
a proximal section that includes a first cylindrical section with a first outer diameter; a second cylindrical section with a second outer diameter that is less than the first diameter; a first transitional section between the first cylindrical section and the second cylindrical section; a collet having one or more shoulders connected to the second cylindrical section, and one or more arms extending proximally from the one or more shoulders; and a distal section having a third cylindrical section with an inner diameter that is greater than the second outer diameter. In a further aspect, the expandable sheath and the dilator are joined by inserting the dilator into the proximal end of the expandable sheath, sliding the proximal section of the trapping mechanism relative to the distal section of the trapping mechanism, compressing one or more arms of the collet by the third cylindrical section when the proximal section of the trapping mechanism is moved in a distal direction relative to the distal section of the trapping mechanism, and trapping the expandable sheath between the one or more arms of the collet and a surface of the first transitional section when the proximal section of the trapping mechanism is moved in a distal direction relative to the distal section of the trapping mechanism.

In a further aspect, the distal end of the expandable sheath is tapered. In any of the above aspects, an internal surface of the distal end of the expandable sheath includes a step feature.

In any of the above aspects, a sheath hub is coupled to the distal end of the expandable sheath; and a dilator hub is coupled to a distal end of the body of the dilator, wherein the dilator hub may have one or more latches wherein the method further comprises locking the dilator hub to the sheath hub.

In any of the above aspects, the expandable sheath may have an expandable frame and a material applied to the frame wherein the material may be formed from a polymer that is optionally a thermoplastic polyurethane. In a further aspect, the frame may be formed from a braided material that is optionally strands of Nitinol.

In a further aspect, the expandable sheath may have a coating applied to the expandable frame and the material, which, in one aspect, may be a lubricious coating. In a further aspect, the trapping mechanism and/or the collet may be formed from Nitinol. In a further aspect, the trapping mechanism may be formed from stainless steel or a polymer.

In a further aspect, the tapered tip of the dilator may be formed from a polymer that may be a polyether block amide.

From the foregoing and with reference to the various figure drawings, those skilled in the art will appreciate that certain modifications can also be made to the present disclosure without departing from the scope of the same. While several examples of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects of the technology. Those skilled in the art will envision other modifications within the scope and spirit of the claims appended hereto.

1. An apparatus, comprising:
an expandable sheath having a proximal end, a distal end, and a lumen running from the proximal end to the distal end;
a dilator comprising a cylindrical body, a tapered tip, and a trapping mechanism between the body and the tapered tip, the trapping mechanism comprising:
a proximal section having:
a first cylindrical section with a first outer diameter;
a second cylindrical section with a second outer diameter that is less than the first diameter;
a first transitional section between the first cylindrical section and the second cylindrical section; and
a collet having one or more shoulders connected to the second cylindrical section, and one or more arms extending proximally from the one or more shoulders; and
a distal section having a third cylindrical section with an inner diameter that is greater than the second outer diameter,
wherein the dilator is configured to be inserted into the proximal end of the expandable sheath,
wherein the proximal section of the trapping mechanism is configured to slide relative to the distal section of the trapping mechanism,
wherein the one or more arms of the collet are configured to be compressed by the third cylindrical section when the proximal section of the trapping mechanism is moved in a distal direction relative to the distal section of the trapping mechanism, and
wherein the trapping mechanism is configured to trap the expandable sheath between the one or more arms of the collet and a surface of the first transitional section when the proximal section of the trapping mechanism is moved in a distal direction relative to the distal section of the trapping mechanism.

2. The apparatus of claim 1, wherein the distal end of the expandable sheath is tapered.

3. The apparatus of claim 1, wherein an internal surface of the distal end of the expandable sheath includes a step feature.

4. The apparatus of claim 1, further comprising:
a sheath hub coupled to the distal end of the expandable sheath; and
a dilator hub coupled to a distal end of the body of the dilator having one or more latches configured to lock the dilator hub to the sheath hub.

5. The apparatus of claim 1, wherein the expandable sheath comprises an expandable frame and a material applied to the frame.

6. The apparatus of claim 5, wherein the material comprises a polymer or a thermoplastic polyurethane.

7. The apparatus of claim 5, wherein the frame comprises a braided material.

8. The apparatus of claim 7, wherein the braided material comprises strands of Nitinol.

9. The apparatus of claim 5, wherein the expandable sheath further comprises a coating applied to the expandable frame and the material.

10. The apparatus of claim 9, wherein the coating is a lubricious coating.

11. The apparatus of claim 1, wherein the trapping mechanism and/or the collet comprises Nitinol.

12. The apparatus of claim 1, wherein the trapping mechanism comprises stainless steel or a polymer.

13. The apparatus of claim 1, wherein the tapered tip of the dilator comprises a polymer or a polyether amid block.

14. A method for advancing a sheath using a dilator, the method comprising:
joining an expandable sheath having a proximal end, a distal end, and a lumen running from the proximal end to the distal end to a dilator comprising a cylindrical body, a tapered tip, and a trapping mechanism between the body and the tapered tip, the trapping mechanism comprising:
a proximal section having:
a first cylindrical section with a first outer diameter; 'a second cylindrical section with a second outer diameter that is less than the first diameter;
a first transitional section between the first cylindrical section and the second cylindrical section; and
a collet having one or more shoulders connected to the second cylindrical section, and one or more arms extending proximally from the one or more shoulders; and
a distal section having a third cylindrical section with an inner diameter that is greater than the second outer diameter,
wherein the expandable sheath and the dilator are joined by inserting the dilator into the proximal end of the expandable sheath,
sliding the proximal section of the trapping mechanism relative to the distal section of the trapping mechanism,
compressing one or more arms of the collet by the third cylindrical section when the proximal section of the trapping mechanism is moved in a distal direction relative to the distal section of the trapping mechanism, and
trapping the expandable sheath between the one or more arms of the collet and a surface of the first transitional section when the proximal section of the trapping mechanism is moved in a distal direction relative to the distal section of the trapping mechanism.

15. The method of claim 14, wherein the distal end of the expandable sheath is tapered.

16. The method of claim 14, wherein an internal surface of the distal end of the expandable sheath includes a step feature.

17. The method of claim 14, wherein a sheath hub is coupled to the distal end of the expandable sheath; and
a dilator hub is coupled to a distal end of the body of the dilator, the dilator hub comprising one or more latches wherein the method further comprises locking the dilator hub to the sheath hub.

18. The method of claim 14, wherein the expandable sheath comprises an expandable frame and a material applied to the frame.

19. The method of claim 18, wherein the material comprises a polymer or a thermoplastic polyurethane.

20. The method of claim 18, wherein the expandable frame comprises a braided material.

21. The method of claim 20, wherein the braided material comprises strands of Nitinol.

22. The method of claim 18, wherein the expandable sheath further comprises a coating applied to the expandable frame and the material.

23. The method of claim 22, wherein the coating is a lubricious coating.

24. The method of claim 14, wherein the trapping mechanism and/or the collet comprises Nitinol.

25. The method of claim 14, wherein the trapping mechanism comprises stainless steel or a polymer.

26. The method of claim 14, wherein the tapered tip of the dilator comprises a polymer or a polyether amide block.
